(19) United States
(12) Patent Application Publication
(10) Pub. No.: US 2016/0023266 A1
(43) Pub. Date: Jan. 28, 2016

(54) DEVICE AND METHOD FOR PRODUCING CLAMPS BENT FROM WIRE SECTIONS

(71) Applicant: ZAHORANSKY AG, Todtnau (DE)

(72) Inventors: Christoph Albrecht, Todtnau-Aftersteg (DE); Philipp Goldmann, Freiburg (DE)

(21) Appl. No.: 14/774,708

(22) PCT Filed: Feb. 21, 2014
(86) PCT No.: PCT/EP2014/000462

§ 371 Date: Sep. 11, 2015

(30) Foreign Application Priority Data
Mar. 16, 2013 (DE) 10 2013 004 652.1

(51) Int. Cl. B21F 45/00; A61B 17/064; B21F 45/16

(52) U.S. Cl. CPC B21F 45/008; B21F 45/16; A61B 17/064; A61B 2017/00526

(57) ABSTRACT

A device (1) for producing clamps (3) bent from wire sections (2), in particular surgical clamps, having a bending device (22) for bending a wire section (2), having a feed device (4) for the wire (5) having a cutting device (6) for separating a wire section (2) from the fed wire (5). The cutting device (6) has two cutting punches (11) for cutting the wire (5). A holding device (14) is provided for gripping and holding the wire section (2) between the two cutting punches (11) before separating the wire section (2) and for positioning the separated wire section (2) on a bending device (22) and for feeding the wire section (2) bent into the clamp to a device (33) for inserting the clamp (3) into a cartridge (34) or a clamp storage (35).

The invention relates to a device and to a method for producing clamps bent from wire sections, in particular surgical clamps, with a bending device for bending a wire section.

Numerous realizations of such devices are known. The problem here, however, is that the wire section can be displaced or twisted when acted upon by the bending device such that the clamps are subject to comparatively large production tolerances. Even when the wire section is severed from a fairly long piece of wire, corresponding positional displacements can occur resulting in measurement inaccuracies.

U.S. Pat. No. 5,060,468 shows and describes a device together with a method where a prefabricated wire section is insertable into a holder. The free ends of the wire section are then acted upon by a severing device and at the same time are bent and shortened. The wire section is certainly held in a clamping manner in this case, which reduces the risk of positional displacements, but as a result of shortening the free ends of the wire section, a large amount of scrap material is generated with each individual clamp.

Consequently, the object consists in creating a device and a method of the type mentioned in the introduction, by way of which clamps with small production tolerances are able to be produced. In addition, no scrap material or only a little scrap material is to accrue.

The solution according to the invention consists with respect to the device in that a feed device for a wire is provided with a cutting device for severing a wire section from the supplied wire, that the cutting device comprises two cutting punches for cutting the wire, and that a holding device is provided for gripping and holding the wire section between the two cutting punches prior to severing the wire section and for positioning the severed wire section on a bending device as well as for supplying the wire section bent to form the clamp to a device for introducing the clamp into a cartridge or a clamp storage.

As the wire is already gripped prior to the severing in the region of the wire section to be severed and is held continuously during the severing and the bending until it is fed to the device for introducing the clamps into a cartridge or a clamp storage without the wire section or the wire section which is bent to form the clamp being released in the meantime, the wire sections can be severed and bent at a repeat accuracy of 1/100 millimeter. Slipping in the longitudinal direction or twisting is reliably avoided. In addition, unnecessary loss of material is avoided as the wire section which is severed by the cutting punches is bent to form the finished clamp without its length being shortened any further.

A preferred embodiment in this case provides that the holding device is realized in a tongs-like manner and comprises a holding region which is movable against a resetting force from a holding position into an open position. This makes it possible to fix the holding device on the wire section in a simple manner and inadvertent release is reliably avoided by means of the resetting force.

The bending device can comprise in particular an anvil as a forming element for the wire section which is mounted in a linearly displaceable manner between two pressing rollers which are each adjustable against a resetting force.

As a result, the wire section is able to be bent to form the clamp in a simple manner. The wire section, in this case, is placed by way of its central region, which forms the clamp back of the finished clamp, onto an end face of the anvil. As a result of the two free ends which protrude on both sides being acted upon, said free ends are bent by the pressing rollers and pressed against the longitudinal sides of the anvil. These bent ends of the wire section consequently form the legs of the finished clamp. The form of the finished clamp can be established as a result of the shaping of the anvil. For example, the end regions of the legs can be additionally bent inward as a result of a corresponding anvil form with a tapering region.

In addition, the bending device can comprise an anvil which comprises stop surfaces for the bent wire legs and pressing rollers which are assigned to said wire legs for shearing the wire legs against one another.

After the wire sections have been bent, the clamp can be placed onto a further anvil in order to deflect the wire sections out of the flat clamp plane and to fold them toward one another. This can be useful in particular in the case of clamps with long legs so that the free ends of the legs do not touch each another during the clamping operation, which can result in an incomplete clamping operation and in an insufficient hold.

It is also possible for the anvil of the bending device to comprise a circle-segment-shaped forming region for the wire section in order to be able to produce correspondingly formed clamps with a rounding.

For uniform and synchronous movement of the holding device and the anvil, it is expedient when the holding device is releasably connectable to the anvil of the bending device.

The feed device can comprise in particular a wire roll as a hopper for the wire to be supplied. This enables a large quantity of wire to be provided in a small space.

In order to eliminate curvatures from the wire unwound from the wire roll and to ensure that the wire is supplied in a straight form in the region of the cutting punches, the feed device can comprise a straightening mechanism with several guide rollers arranged on both sides of the wire for aligning the wire unwound from the wire roll.

Also for straightening the unwound wire, the feed device can comprise two clamping elements which are linearly displaceable in the advancing direction of the wire. A section of the wire is held in clamping manner by the two clamping elements which are spaced apart with respect to one another. One of the clamping elements is then moved away from the other clamping element by a small distance as a result of a linear displacement such that the spacing between the two clamping elements is increased. Where applicable, both clamping elements can also be moved in opposite directions. As a result, the wire section clamped between the clamping elements is straightened and possible curvatures eliminated.

The cutting punches can preferably comprise in each case an oblique cutting edge. As a result, the wire section can be severed such that the free ends of the wire section comprise a corresponding lead-in chamfer. Additional cutting operations at the ends of the wire section are consequently not necessary.

In order to be able to vary the orientation of the cutting edge in a simple manner, it is advantageous when the cutting punches are mounted in each case in a punch guide which is pivotable about an axis oriented in the longitudinal direction of the wire and/or can be arranged rotated by 180° with reference to the longitudinal direction of the wire.

A least one processing device for the wire or the wire section can be arranged upstream and/or downstream of the bending device. For example, material weakening can be introduced into the wire prior to the bending in order to enable simpler bending in this manner at said positions. In the case of a corresponding requirement for the finished clamp, the free ends of the wire section can also be processed, for example ground, sharpened or twisted. The processing device can also be realized for installing a marking, for example a bar code, a date code or an identity code. The marking, in this case, can be effected, for example, by means of laser engraving.

It can be expedient when a control device is provided for the bent wire section. In this case, in particular the form of the bent clamp and/or the spring force of the clamp legs can be checked.

A preferred embodiment provides that the device for introducing the clamp into a cartridge or a clamp storage mean comprises a slide for acting upon the clamp on the clamp rear thereof. As consequently the free ends of the clamp legs, which represent the most sensitive part of the clamp, are not acted upon, damage to them when the clamp is introduced is avoided in a reliable manner.

In this case, it is expedient when the slide is adapted to the shape of the wire section in the impact region. This enables reliable guiding of the clamp by the slide.

It can additionally be expedient when the slide is thinner in the impact region than the wire section to be acted upon. For example, the slide can be thinner than the wire section by between 0.01 mm and 0.1 mm. As a result, the slide can push the clamps into a cartridge or a clamp storage without itself impacting against the cartridge or the clamp storage.

In a preferred manner, a drive unit is provided for linear displacement of the slide.

The drive unit can comprise a force sensor and a control unit for controlling the slide in dependence on the sensor signals. Too strong an application of force at the drive unit can indicate, for example, a wrongly positioned clamp which cannot be introduced correctly into the cartridge or a clamp storage. In such a case, the drive unit can be stopped by the control unit in order to avoid damage to the device.

It is also possible for the drive unit to comprise a force-controlled coupling unit. In the case of too high an application of force, the coupling unit can disengage such that the slide is no longer driven. The coupling unit can be formed, for example, by spring clamping which slips in the event of too high a load.

In a preferred manner, the device for introducing the clamp into a cartridge or a clamp storage can comprise a housing with a guide for the slide and an insertion opening for inserting in each case one clamp.

In order to be able to fill the individual clamp receivers in a clamp storage or of a cartridge one after another by way of the device for introducing the clamps, it is expedient when a positioning device for positioning a clamp storage or a cartridge is provided upstream of the output-side end of the device for introducing the clamp into a cartridge or a clamp storage. Consequently, a clamp receiving means can be positioned in each case in front of the stationary device in order to introduce one clamp in each case into the clamp receiving means by way of the slide.

The individual components of the device (feed device, cutting device, holding device, bending device and device for introducing the clamps into a cartridge or a clamp storage) can be arranged in individual processing stations spaced apart from one another. In a preferred manner, the individual components of the device are arranged, however, in one common processing station. This enables the entire device to be designed in a compact and space-saving manner.

With regard to the method, the invention is characterized in that the wire section is cut off a wire and the free ends of the wire section are bent to form wire legs, that the wire section that is bent to form a clamp is introduced into a cartridge or a clamp storage, and that the wire section is gripped prior to severing and is held constantly during the severing and the bending.

The advantages that have already been explained in the case of the description of the device according to the invention are produced in this case.

The wire is preferably unrolled from a wire roll.

In this case, it is advantageous when the wire is aligned and/or stretched prior to the severing. As a result, possible bends are eliminated and the wire is aligned precisely in a straight manner prior to severing and bending to form the clamp, as a result of which the desired precision of the clamps is able to be achieved.

The bending of the wire section to form the clamp can be effected in a good and precise manner when the severed wire section is placed by way of its central region, which forms the clamp back of the finished clamp, onto an anvil and the anvil is moved through between two pressurized pressing rollers and, as a result, the wire regions protruding beyond the anvil are bent.

It can be expedient when the bent wire legs are bent out of the clamp plane and are sheared against one another. In particular in the case of clamps with long legs, this reliably avoids the wire legs touching each other at their ends and thus being able to impair reliable clamping.

The wire section can be processed upstream and/or downstream of the bending process. For example, material weakening can be introduced into the wire section or the wire ends can be ground or sharpened. It is also possible to install markings onto the wire section.

The invention is explained in more detail below by way of the drawings, in which, in part in schematic representations:

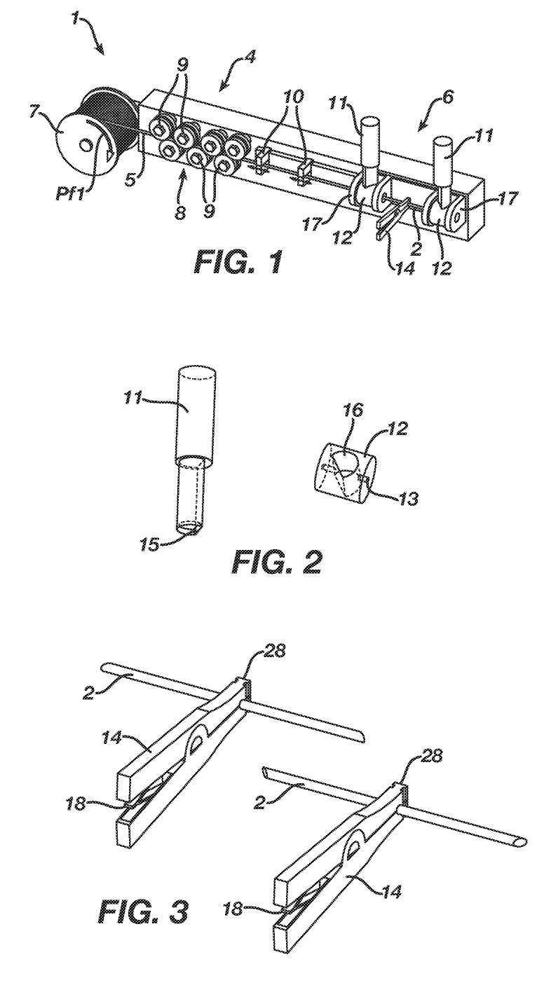

FIG. 1 shows a feed device with a straightening mechanism, clamping elements and a cutting device,

FIG. 2 shows a cutting punch and a punch guide means,

FIG. 3 shows wire holding devices with in each case a wire section held thereon,

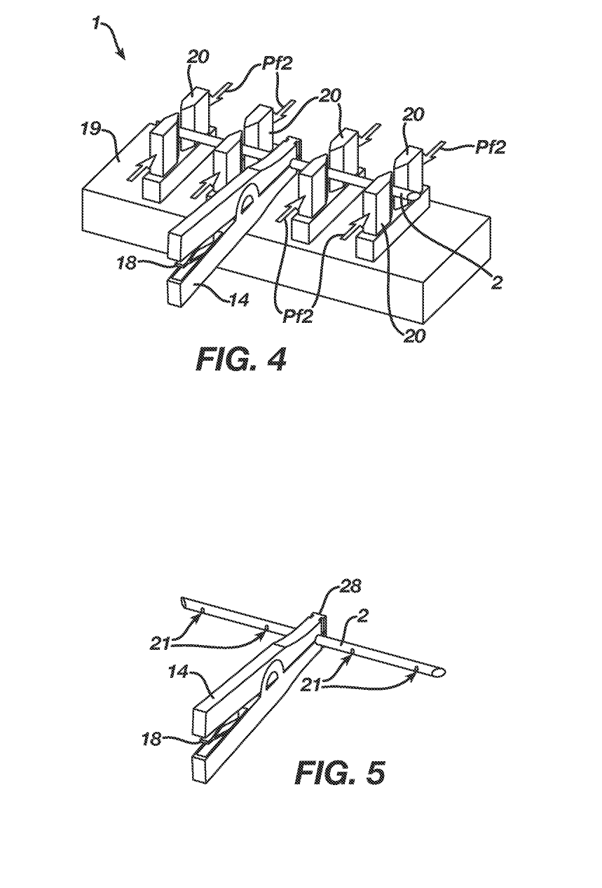

FIG. 4 shows a processing device for processing a wire section,

FIG. 5 shows a wire holding device with a wire section that has been processed in the processing device from FIG. 4,

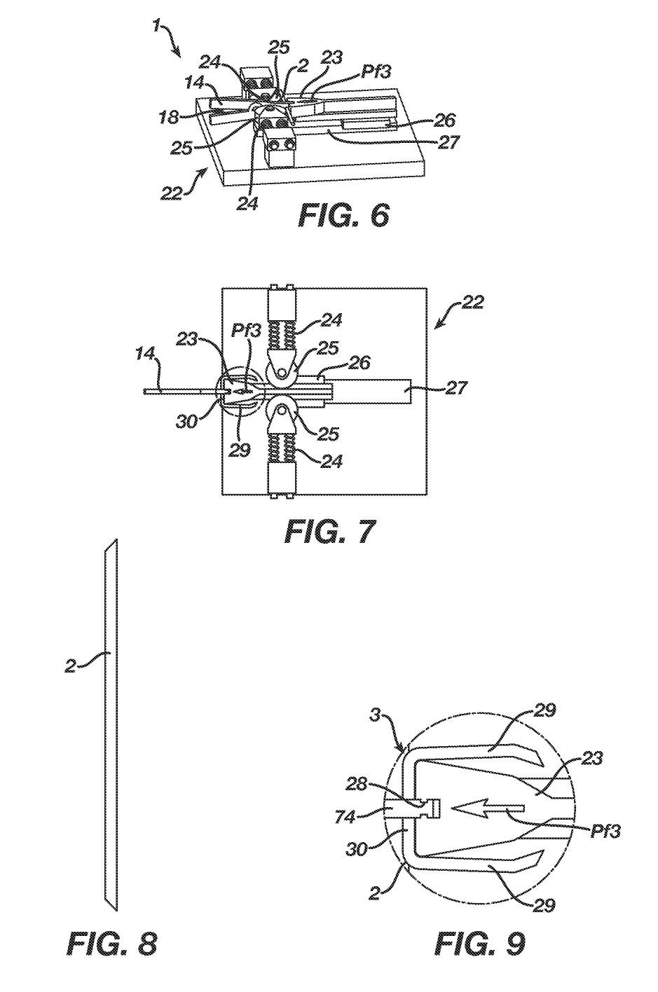

FIG. 6 shows a perspective view of a bending device for bending a wire section,

FIG. 7 shows a top view of a bending device for bending a wire section,

FIG. 8 shows a bent wire section,

FIG. 9 shows a detail of the representation of the wire section that has been bent according to FIG. 7,

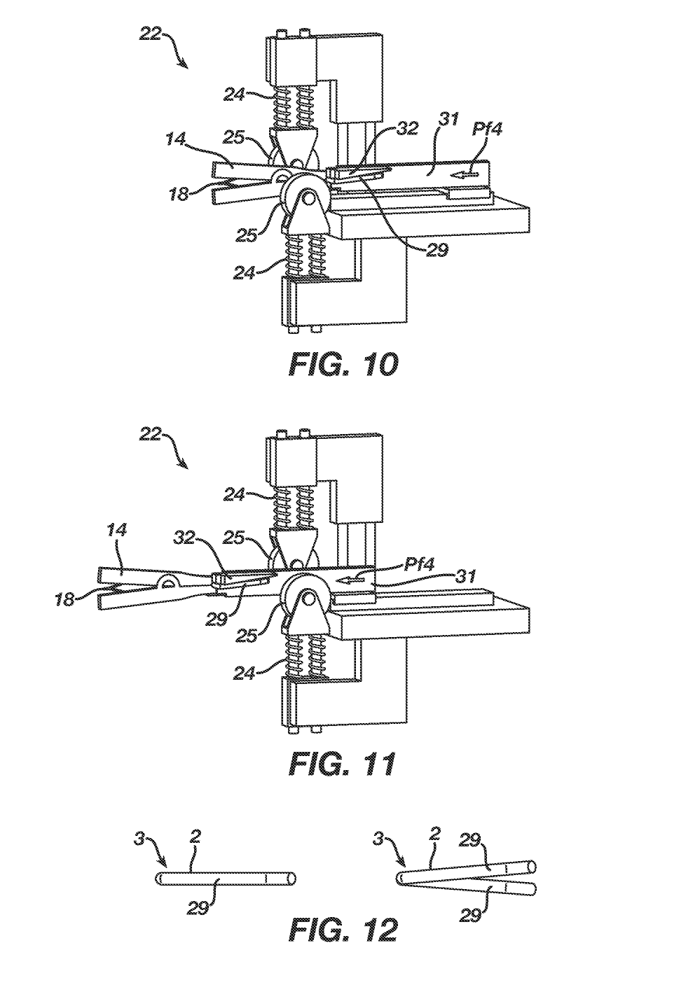

FIG. 10 and FIG. 11 show a bending device for severing the bent wire legs,

FIG. 12 shows side views of a clamp prior to and after the severing of the wire legs,

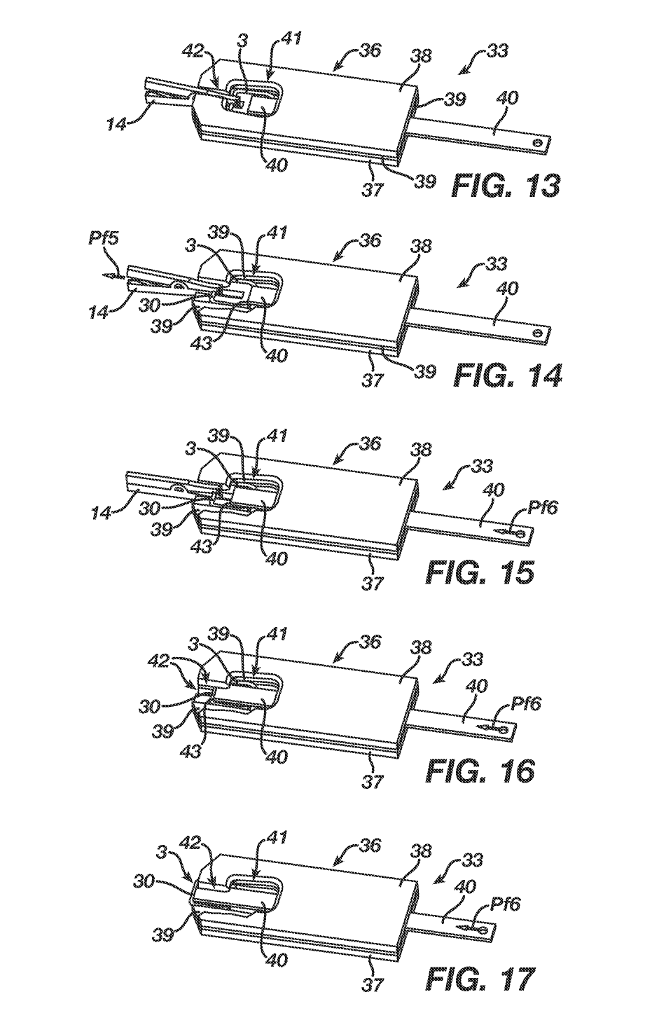

FIG. 13 to FIG. 17 show in each case a device for introducing a clamp into a cartridge or into a clamp storage,

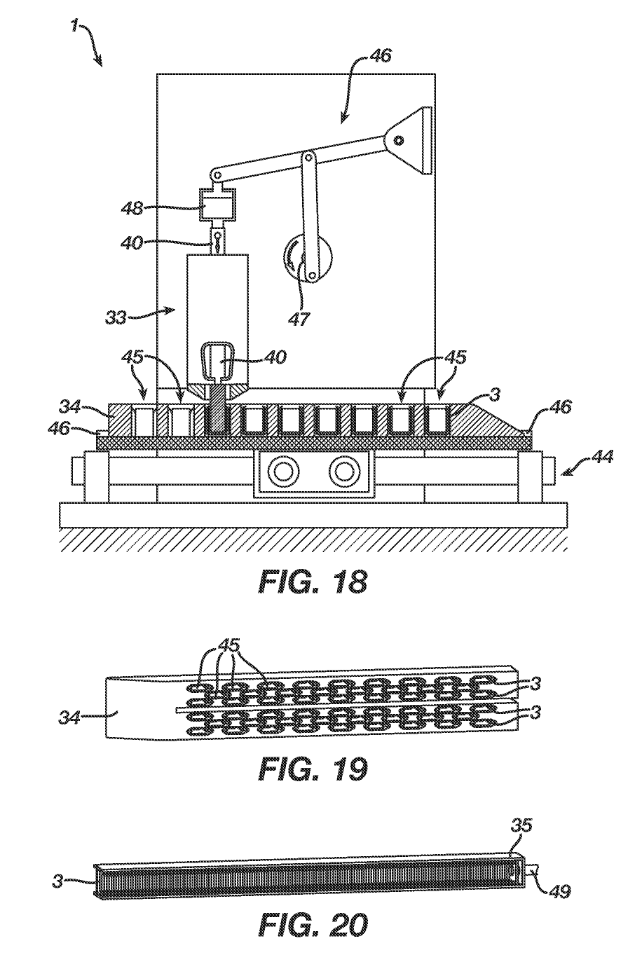

FIG. 18 shows a device for introducing a clamp into a cartridge with a cartridge which is positionable in front of said device,

FIG. 19 shows a cartridge filled with clamps,

FIG. 20 shows a clamp storage filled with clamps,

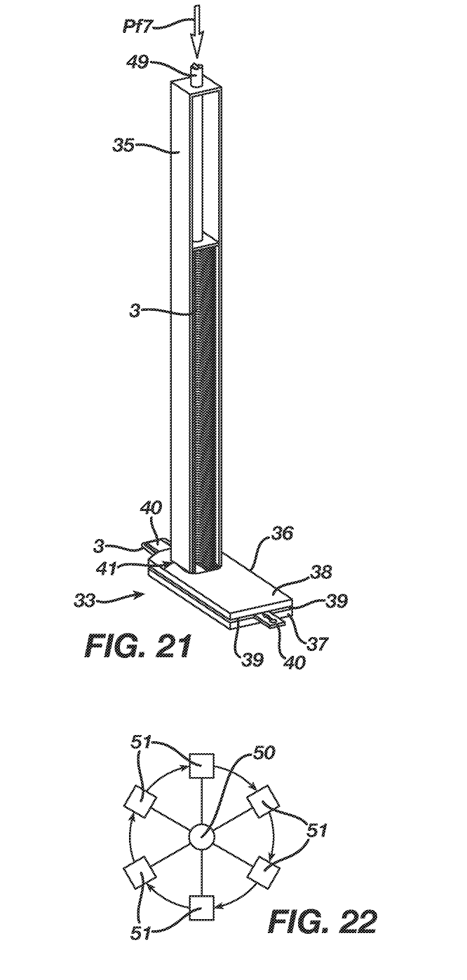

FIG. 21 shows a clamp storage as a hopper for feeding clamps to a device for introducing a clamp into a cartridge, and

FIG. 22 shows a schematic representation of the individual processing stations of the device.

A device, given the overall reference of 1, for producing clamps 3 which are bent from wire sections 2 comprises, according to FIG. 1, a feed device 4 for supplying wire 5 to a cutting device 6. A wire roll 7, from which the wire 5 is unwound as a result of rotating the wire roll 7 in the direction of the arrow Pf1, is provided as a supply. The wire can consist, in particular, of titanium or a titanium-aluminum-vanadium alloy.

In order to eliminate curvatures in the unwound wire 5, the feed device 4 comprises a straightening mechanism 8 with several guide rollers 9 which are arranged on both sides of the wire 5. Possible curvatures of the wire 5 are eliminated when the wire 5 runs through the guide rollers 9 which are arranged alternately on both sides of the wire 5.

After running through the straightening mechanism 8, the further conveyed wire 5 is gripped by two clamping elements 10. The left-hand clamping element 10 in FIG. 1 holds the wire 5 in this case, whilst the right-hand clamping element 10 is moved by a small distance to the right, away from the wire roll 7. As a result, the wire 5 is stretched in the region between the clamping elements 10 and any bends possibly remaining are eliminated as a result.

Both clamping elements 10 are then moved in a synchronous manner to the right such that a section of the wire 5 that was stretched beforehand is pushed to the right of the clamping elements 19 into the region of the cutting device 6. The clamping elements 10 are then opened and moved back into their starting position such that that they are able to hold a further region of the wire 5, which has run through the guide rollers 9 beforehand, in a clamping manner.

The cutting device 6 comprises two cutting punches 11 which are mounted in each case in a punch guide means 12, the wire 5 being guided in each case through an opening 13 (FIG. 2) in the punch guide means 12.

Once the wire 5 has been fed to the cutting device 6, the wire 4 is gripped between the two cutting punches 11 by way of a holding device 14. Only then is the section of the wire 5 between the two cutting punches 11 severed from the supplied wire 5 as a result of actuating the cutting punch 11. As a result of the form of the cutting edge 15 of the cutting punch 11 (FIG. 2), the wire 5 is cut twice by the left-hand cutting punch 11 in FIG. 1. If the wire 5 is only advanced such that its free end is arranged inside the receiving opening 16 for the cutting punch 11 of the right-hand punch guide 12, the wire 5 is cut only at one position according to FIG. 1 by the right-hand cutting punch 11. As a result, loss of material as a result of cutting waste is avoided. If the wire 5, however, is pushed further into the right-hand punch guide 12, the wire 5 is also cut by the right-hand cutting punch 11 at two positions. However, this does not have a negative effect on the quality of the severed wire section 2 (FIG. 3). The geometry of the left-hand cutting punch 11 could also be provided such that the cutting edge of the wire 5 remaining on the left of said cutting punch 11 already corresponds to the desired development of a wire section 2 to be subsequently severed. It would then be possible to dispense with the right-hand cutting blade 11. However, this can result in dimensional inaccuracies in the severed wire sections 2.

The wire 5 can be guided by additional guide elements (not shown) inside the feed device 4. The position of the punch guide means 12 and consequently of the cutting punch 11 can be adjusted in the longitudinal direction of the wire 5 in order, on the one hand, to be able to cut off variously long wire sections 2 and in order, on the other hand, to enable unobstructed removal of a severed wire section 2 from the region between the two punch guide means 12.

FIG. 2 shows a cutting punch 11, its wedge-shaped cutting edge 15 being recognizable. A punch guide means 12 with the receiving opening 16 for the cutting punch 11 and the opening 13 for the wire 5 is shown on the other side.

The punch guide means 12 are held so as to be releasable in each case in holders 17 (FIG. 1). As a result of changing the side the punch guide means 12 is inserted into the holder 17, the orientation of the cutting edge of the severed wire section 2 can be varied, as is clarified by way of the two wire sections 2 shown in FIG. 3.

When other cutting angles or other cutting forms are desired, the punch guide means 12 can be exchanged by correspondingly developed examples.

It is also easy to see in FIG. 3 that the holding device 14 is realized in a tongs-like manner and the holding region that holds the wire section 2 is movable against the resetting force of a tension spring 18 from the holding position shown into an open position.

Once the wire section 2 has been severed, it is supplied with the holding device 14 to a processing device 19 (FIG. 4) which is also a component part of the overall device 1. At the processing station 19, pinching or material weakening 21 is introduced into the wire section 2 (FIG. 5) using impact wedges 20. To this end, the impact wedges 20 are introduced to the wire section 2 according to the arrows Pf2 from FIG. 4 at a corresponding pressing force. As a result of the material weakening 21, the wire section 2 can be better bent at said points. The processing device 19 can also comprise further processing tools (not shown), for example for grinding or sharpening the free wire ends. These different processing processes can be carried out on one single processing device 19, or several processing devices 19 can be provided at which the wire section 2 is positioned one after another.

According to FIGS. 6, 7 and 9, the wire section 2 is then supplied to a bending device 22. The bending device 22 comprises an anvil 23 as a shaping element for the wire section 2. The anvil 23 is mounted so as to be linearly displaceable between two pressing rollers 25 which are each adjustable against the resetting force of compression springs 24. According to FIG. 6, the anvil 23 is mounted on a guide rail 27 with a guide carriage 26 for this purpose. So that the wire section 2 is also held by the holding device 14 during the bending operation and the holding device 14 and the anvil 23 are moved in a synchronous manner, the holding device 14 and the anvil 23 are connectable to one another in a releasable manner. For this purpose, the holding device 14 comprises a profiled connecting end 28 (FIG. 9) which is insertable into a correspondingly counter-profiled recess in the anvil 23.

As shown in FIG. 6, the holding device 14 with the wire section 2 is docked onto the anvil 23 which is positioned upstream of the pressing rollers 25. According to the arrow Pf3, the anvil 23 with the holding device 14 is pulled through between the pressing rollers 25. As a result, the ends of the wire section 2 which protrude beyond the end face of the anvil 23, against which the wire section 2 abuts, are bent by the pressing rollers 25 and pressed against the side walls of the anvil 23. As a result of the form of the anvil 23 and of the material characteristics of the wire section, the bent wire legs 29, once they have been pulled through by the pressing rollers 25, spring back again a little, which can be seen clearly in FIG. 7 and in particular in the representation of a detail according to FIG. 9. The region of the wire section 2 which abuts against the end face of the anvil 23 forms the clamp back 30 of the finished clamp 3.

FIG. 8 shows the non-bent wire section 2 prior to bending.

According to FIGS. 10 and 11, the bending device 22 can comprise a further anvil 31 which comprises stop surfaces 32 for the bent wire legs 29 and pressing rollers 25 assigned to said bent wire legs for shearing the wire legs 29 against one another. The wire section 2 with the bent wire legs 29, continuing to be held by the holding device 14, is docked onto the further anvil 31, and said further anvil is moved according to arrow Pf4 past the pressing rollers 25 which are mounted with compression springs 24. As a result of the wire legs 28 being acted upon by the pressing rollers 25 and as a result of the development of the stop surfaces 32 of the anvil 31 which act as a delimiting means, the wire legs 29 are deflected in opposite directions out of their original plane. The wire legs 29 also spring back elastically after said bending operation.

FIG. 12 shows a side view of the wire section 2 which is bent to form the clamp 3 prior to (on the left) and after (on the right) the bending of the device according to FIGS. 10 and 11. The folding of the wire legs 28 prevents the free ends of the clamp 3 being able to meet during the clamping operation.

FIGS. 13 to 17 show in each case a device 33 for introducing a clamp 3 into a cartridge 34 (FIG. 19) or a clamp storage 35 (FIG. 20). The device 33 comprises, in this case, a housing 36 with a base plate 37, a cover plate 38 and intermediate plates 39 lying in between. The intermediate plates 39 form side guide means for a slide 40 which is mounted so as to be longitudinally displaceable in the housing 36. A clamp 3 is placed into the device 33 by means of an opening 41 (FIG. 13). The clamp 3, in this case, is still held by the holding device 14. The base plate 37 and the cover plate 38 comprise on the end-face end of the housing 33 in each case a recess 42 for receiving the holding device 14. Once the clamp 3 with the holding device 14 has been introduced into the device 33 through the opening 41, the clamp 3 rests on the base plate 37 and is guided at the side by the intermediate plates 39. As a result of moving the holding device 14 in the direction of the arrow Pf5 (FIG. 14), the clamp 3 is pulled under the cover plate 38 with its clamp back 30 such that the clamp 3 is also secured between the base plate 37 and the cover plate 38.

FIGS. 14 to 17 show the cover plate 38 in a partially broken manner for a better overview.

According to FIG. 15, the holding device 14 is moved into its open position and removed from the clamp 3. The slide 40 is displaced in the direction of the clamp 3 according to arrow Pf6 until the slide 40 acts upon the clamp back 30 of the clamp 3 (FIG. 16). The impact end 43 of the slide 40, in this case, is adapted to the form of the clamp back 30 such that the clamp 3 can be acted upon and displaced without any risk of damage.

As a result of displacing the slide 40 further in a linear manner, the clamp 3 is displaced out of the housing 36 and the device 33 (FIG. 17) and into a cartridge according to FIG. 19 or a clamp storage according to FIG. 20.

Realizations would also be conceivable where a clamp 3 is held by the holding device 14 such that the holding device 14 is pivoted upward by 90° in relation to the representation shown. The base plate 37 and the cover plate 38 could then be realized without recesses 42.

It would also be conceivable to introduce the clamp 3 from the left side of the housing 36 in FIGS. 13 to 17, through which side the clamp 3 is subsequently displaced again out of the housing 36 with the slide 40. In the case of such an embodiment, the opening 41 in the cover plate 38 would not be necessary. In this case, however, care must be taken to ensure that the clamp 3 is guided in a precise manner in order not to damage the free ends of the clamp 3 which are then at the front in the advancing direction when the clamp 3 is inserted into the housing 36.

Pushing a clamp 3 into a cartridge 34 is also shown in FIG. 18. A cartridge 34 with several clamp receiving means 45 for receiving the clamps 3 is arranged below the device 33 for introducing the clamps 3. By means of a positioning device 44, each one of the clamp receiving means 45 is positionable in front of the device 33. The cartridge 34, which is fixed in a releasable manner on the positioning device 44 by a clamping device 46, is positionable inside a plane by means of the positioning device 44 which is realized as a multi-axis system. Consequently, in each case one clamp receiving means 45 can be moved in front of the device 33 in order to fill all the clamp receiving means 45 of the cartridge 34 one after another with in each case one clamp 3 (FIG. 19).

A drive unit 46, which converts the rotary movement of a drive motor 47 into a linear movement for displacing the slide 40, is provided according to FIG. 18. The drive unit 46 comprises a force sensor 48 in the region of the slide 40. In the case of a wrongly positioned cartridge 34 or a falsely or wrongly positioned clamp 3, increased forces occur at the force sensor 48. The drive unit 46 can be stopped by means of a control unit (not shown) in order to avoid further damage.

FIG. 19 shows a cartridge 34 filled with clamps 3, as can be used, for example, in the medical sector for clamping wounds.

As an alternative to this, clamps 3 can also be inserted into a clamp storage 35 according to FIG. 20. The clamps 3 can be stored for subsequent use in such a clamp storage 35.

For example, a clamp storage 35, as shown in FIG. 21, can itself be used as a feed hopper for a device 33 for introducing a clamp 3 into a cartridge 34. A slide pin 49, which is acted upon with pressure (arrow pf7), pushes the clamps 3 located in the clamp storage 35 downward and guides in each case one clamp 3 into the insertion opening 41 of the housing 36, where the clamp 3 is grasped by the slide 40 and inserted into a cartridge which is not shown in FIG. 21.

FIG. 22 shows a schematic representation of the processing sequence with the individual processing steps that have been described beforehand and the individual processing stations. A central control means 50, in this case, controls the sequences in the individual processing stations 51. The individual processing stations 51, for example for severing the wire section or for bending the wire section, in this case, do not have to be located spatially separate from one another as symbolized in FIG. 22, but can also be combined in one common, spatially compact processing station.

Individual processing stations can also be effected at the same time, for example a clamp 3 can be inserted into a cartridge 34 whilst a new wire section 2 is being cut off and bent.

Twin machines, where individual processing steps are effected on multiple devices in parallel, are also conceivable.

What is claimed:

1. A device (1) for producing clamps (3) bent from wire sections (2), in particular surgical clamps, said device having a bending device (22) for bending a wire section (2), characterized in that a feed device (4) for a wire (5) is provided with a cutting device (6) for severing a wire section (2) from the supplied wire (5), in that the cutting device (6) comprises two cutting punches (11) for cutting the wire (5), and in that a holding device (14) for gripping and holding the wire section (2) between the two cutting punches (11) prior to severing the wire section (2) and for positioning the severed wire section (2) on a bending device (22) as well as for supplying the wire section bent to form the clamp (3) to a device (33) for introducing the clamp (3) into a cartridge (34) or a clamp storage (35).

2. The device as claimed in claim 1, characterized in that the holding device (14) is realized in a tongs-like manner and comprises a holding region which is movable against a resetting force from a holding position into an open position.

3. The device as claimed in claim 1 or 2, characterized in that the bending device (22) comprises an anvil (23) as a forming element for the wire section (2) which is mounted in a linearly displaceable manner between two pressing rollers (25) which are each adjustable against a resetting force.

4. The device as claimed in one of claims 1 to 3, characterized in that the bending device (22) comprises an anvil (31) which comprises stop surfaces (32) for bent wire legs (29) and pressing rollers (25) which are assigned to said wire legs for shearing the wire legs (29) against one another.

5. The device as claimed in claim 3, characterized in that the anvil (23) of the bending device (22) comprises a circle-segment-shaped forming region for the wire section (2).

6. The device as claimed in one of claims 3 to 5, characterized in that the holding device (14) is releasably connectable to the anvil (23, 31) of the bending device (22).

7. The device as claimed in one of claims 1 to 6, characterized in that the feed device (4) comprises a wire roll (7) as a supply for the wire (5) to be supplied.

8. The device as claimed in one of claims 1 to 7, characterized in that the feed device (4) comprises a straightening mechanism (8) with several guide rollers (9) arranged on both sides of the wire (5) for aligning the wire (5) unwound from the wire roll (7).

9. The device as claimed in one of claims 1 to 8, characterized in that the feed device (4) comprises two clamping elements which are linearly displaceable in an advancing direction of the wire.

10. The device as claimed in one of claims 1 to 9, characterized in that the cutting punches (11) comprise in each case an oblique cutting edge (15).

11. The device as claimed in one of claims 1 to 10, characterized in that the cutting punches (11) are mounted in each case in a punch guide (12) which is pivotable about an axis oriented in a longitudinal direction of the wire and/or can be arranged rotated by 180° with reference to the longitudinal direction of the wire.

12. The device as claimed in one of claims 1 to 11, characterized in that at least one processing device (19) for the wire (5) or the wire section (2) is arranged upstream and/or downstream of the bending device (22).

13. The device as claimed in one of claims 1 to 12, characterized in that a control device is provided for the bent wire section (2).

14. The device as claimed in one of claims 1 to 13, characterized in that the device (33) for introducing the clamp (3) into a cartridge (34) or a clamp storage (35) comprises a slide (40) for acting upon the clamps (3) on the clamp rear (30) thereof.

15. The device as claimed in claim 14, characterized in that the slide (40) is adapted to the shape of the wire section (2) in an impact region.

16. The device as claimed in claim 14 or 15, characterized in that the slide (40) is thinner in the impact region than the wire section (2) to be acted upon.

17. The device as claimed in one of claims 14 to 16, characterized in that a drive unit (46) is provided for a linear displacement of the slide (40).

18. The device as claimed in claim 17, characterized in that the drive unit (46) comprises a force sensor (48) and a control unit for controlling the slide (40) in dependence on sensor signals.

19. The device as claimed in claim 17, characterized in that the drive unit (46) comprises a force-controlled coupling unit.

20. The device as claimed in one of claims 1 to 19, characterized in that the device (33) for introducing the clamp (3) into a cartridge (34) or a clamp storage (35) comprises a housing (36) with a guide for the slide (40) and an insertion opening (41) for inserting in each case one clamp (3).

21. The device as claimed in one of claims 1 to 20, characterized in that a positioning device (44) is provided for positioning a clamp storage (35) or a cartridge (34) in front of the output-side end of the device (33) for introducing the clamp (3) into a cartridge (34) or a clamp storage (35).

22. The device as claimed in one of claims 1 to 21, characterized in that the individual components of the device (1) are arranged in a common processing station.

23. A method for producing clamps (3), in particular surgical clamps, where the free ends of a straight wire section (2) are bent to form wire legs (29), characterized in that the wire sections (2) are cut off a wire (5) and the free ends of the wire section (20) are bent to form wire legs (29), in that the wire section (2) that is bent to form a clamp is introduced into a cartridge (34) or clamp storage (35), and in that the wire section (2) is gripped prior to severing and is held constantly during the severing and the bending.

24. The method as claimed in claim 23, characterized in that the wire (5) is unwound from a wire roll (7).

25. The method as claimed in claim 23 or 24, characterized in that the wire (5) is aligned and/or stretched prior to the severing.

26. The method as claimed in one of claims 23 to 25, characterized in that the severed wire section (2) is placed by way of its central region, which forms the clamp back (30) of the finished clamp (3), onto an anvil (23) and the anvil (23) is moved through between two pressurized pressing rollers (25) and, as a result, the wire regions protruding beyond the anvil (23) are bent.

27. The method as claimed in one of claims 23 to 26, characterized in that the bent wire legs (29) are bent out of the clamp plane and are sheared against one another.

28. The method as claimed in one of claims 23 to 27, characterized in that the wire section (2) is processed prior to and/or after the bending process.
